(19) United States
(12) Patent Application Publication
(10) Pub. No.: US 2025/0388873 A1
(43) Pub. Date: Dec. 25, 2025

(54) METHOD FOR THE PURIFICATION OF RECOMBINANT ADENOVIRUS VECTORS

(71) Applicant: Ferring International Center S.A., Saint-Prex (CH)

(72) Inventor: Minna Hassinen, Kuopio (FI)

(21) Appl. No.: 19/209,931

(22) Filed: May 16, 2025

Related U.S. Application Data
(60) Provisional application No. 63/648,225, filed on May 16, 2024.

(30) Foreign Application Priority Data
May 16, 2024 (EP) 24305766.8

(51) Int. Cl. C12N 7/02; A61K 35/761; A61K 48/00; C12N 15/861

(52) U.S. Cl. CPC C12N 7/02; A61K 35/761; A61K 48/005; C12N 15/861; C12N 2710/10343; C12N 2710/10351; C12N 2810/852

(57) ABSTRACT

The present disclosure relates to a method for the purification of such a recombinant adenovirus vector.

### CROSS REFERENCE TO RELATED APPLICATIONS

This application claims priority to U.S. Provisional Application No. 63/648,225, filed May 16, 2024, and EP Application Serial No. 24305766.8, filed May 16, 2024, each of which are hereby incorporated by reference in their entirety.

### FIELD OF THE DISCLOSURE

The present disclosure relates to a method for the purification of a recombinant adenovirus vector.

### BACKGROUND OF THE DISCLOSURE

Recombinant adenovirus vectors have emerged as a potent therapeutic means to treat a number of diseases, including cancers. For example, Nadofaragene firadenovec (otherwise referred to as Adstiladrin), is a gene therapy product approved by the U.S. Food and Drug Administration for the treatment of adult patients with high-risk Bacillus Calmette-Guérin (BCG)-unresponsive non-muscle invasive bladder cancer (NMIBC) with carcinoma in situ (CIS) with or without papillary tumors. Such gene therapy vector has proven to be effective to treat subjects in need thereof. The present disclosure provides improved methods for the purification of recombinant adenovirus vectors, compatible with the clinical demand.

### SUMMARY OF THE DISCLOSURE

As mentioned above, the present disclosure provides methods for the purification of recombinant adenovirus vectors. Implementation of the method disclosed herein results in the provision of vectors suitable for administration to subject in need thereof.

More specifically, the disclosure relates to a method for the purification of a recombinant adenovirus vector, comprising submitting a preparation of recombinant adenovirus vectors to two-step anion exchange chromatography; and recovering the recombinant adenovirus vectors from the two-step anion exchange chromatography elute.

### DETAILED DESCRIPTION

It is herein described effective methods for the purification of recombinant adenovirus vectors. This method achieves high yields of high-quality vectors of clinical grade, that can be administered to a mammalian subject, such as a human subject in need thereof.

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. Likewise, the term “include” and its grammatical variants are intended to be non-limiting, such that recitation of items in a list is not to the exclusion of other like items that can be substituted or added to the listed items. The present disclosure contemplates other embodiments “comprising,” “consisting of” and “consisting essentially of,” the embodiments or elements presented herein, whether explicitly set forth or not.

The singular forms “a,” “and” and “the” include plural references unless the context clearly dictates otherwise. Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Units, prefixes, and symbols are denoted in their Système international d'unités (SI) accepted form. Unless otherwise indicated, nucleic acids are written left to right in 5′ to 3′ orientation; amino acid sequences are written left to right in amino to carboxy orientation.

Groupings of alternative elements or embodiments of the disclosure disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is herein deemed to contain the group as modified thus fulfilling the written description of all Markush groups used in the appended claims.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±20%, in some embodiments ±10%, in some embodiments ±5%, in some embodiments #1%, in some embodiments ±0.5%, and in some embodiments ±0.1% from the specified amount, as such variations are appropriate to perform the disclosed methods.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

### 2. Recombinant Adenovirus Vectors

The disclosure relates to the purification of recombinant adenovirus vectors. The recombinant adenovirus vectors described herein are suitable to deliver a nucleic acid of interest to a cell of a mammalian subject and express the product encoded by said nucleic acid of interest into or from said cell.

In a particular embodiment, the recombinant adenovirus vector may be replication-competent (e.g., meaning that the virus is capable of infecting cells and replicating to produce additional infectious virus) or replication-defective, production of replication-defective adenovirus vectors being preferred. In yet another embodiment, it is herein described the production of recombinant adenovirus serotype 5 (rAd5) vectors, in particular replication-defective rAd5 vectors.

In a particular embodiment, the nucleic acid of interest encodes a therapeutic product and thus the recombinant adenovirus vector is useful for gene therapy. The therapeutic product may be a therapeutic protein or a therapeutic RNA (e.g. a siRNA, miRNA or shRNA), in particular a therapeutic protein. In a further particular embodiment, the therapeutic protein is suitable to treat cancer, in particular bladder cancer, such as non-muscle invasive bladder cancer (NMIBC) or muscle invasive bladder cancer (MIBC). In a further embodiment, the therapeutic protein is human interferon, such as a human Type 1 or Type 2 interferon, in particular human Type 1 interferon, such as human interferon α or β, in particular human interferon α, more particularly interferon α2, even more particularly human interferon α2b.

In addition to said nucleic acid of interest, the genome of the recombinant adenovirus vector comprises one or more regulatory elements suitable to effectively express the nucleic acid of interest in or from the target cell. Such regulatory elements include, without limitation, promoters, enhancers, silencers and/or insulators.

According to a particular embodiment, the recombinant adenovirus vector is a replication-defective rAd5 vector encoding human interferon α2b. Said vector may be constructed with the human gene in an expression cassette which replaces the E1a, E1b and pIX regions at the 5′ end of the adenovirus genome, as is known in the art. Such vector construction can be performed by the skilled person, using standard DNA manipulation techniques.

In a further particular embodiment, the replication-defective rAd5 vector encoding human interferon α2b is nadofaragene firadenovec.

### 3. Production of Recombinant Adenovirus Vectors

Production of recombinant adenovirus vectors have been described previously, for example in WO2016/048556.

The production may in particular be carried out according to a method comprising the following steps:
a) inoculating cells into a bioreactor;
b) infecting said inoculated cells with said recombinant adenovirus vector;
c) lysing the cells;
d) performing a harvest step of recombinant adenovirus vectors from said lysed cells thereby obtaining a bulk harvest of recombinant adenovirus vectors; and
e) filtering said bulk harvest thereby obtaining a filtered bulk harvest of recombinant adenovirus vectors.

### Producer Cells

Inoculated cells may be used as producer cells. As will be apparent herein, production of the recombinant adenovirus vector is carried out after infection of the cells with the recombinant adenovirus vector. Thus, the cells used for production of the recombinant adenovirus vector are permissive to infection by said recombinant adenovirus vector and contain the elements required to propagate the recombinant adenovirus vector after infection. A suitable producer cell is selected depending on the desired recombinant adenovirus vector to be produced. Many therapeutic adenoviral vectors, such as rAd5 vectors, are non-replicating (or replication deficient) whereby the genome is deleted in the E1 region to provide space for alternate gene expression cassettes. The E1 region encodes proteins necessary for the expression of the other early and late genes, hence initiating the viral life cycle. Therefore, when the E1 region is replaced with an expression cassette to produce the gene product that is useful in therapy, such as cytokines, suicide genes, antigens and antibodies, a producer cell line containing adenovirus E1 sequences is required to complement for this region. Therefore, in a particular embodiment in which the recombinant adenovirus vector lacks the adenovirus E1 region, the producer cell used in the practice of the present disclosure contains adenovirus E1 sequences. For example, the cells that can be used to produce the recombinant adenovirus vector can be selected from HEK293, 293T, or any HEK293 related cell line, Per.C6 or SL0036 cells. In a particular embodiment, HEK293 or HEK293T cells are used, in particular HEK293 cells.

### Inoculation of the Bioreactor

Inoculation of the bioreactor in step a) of the production method may be done with cells expanded from an initial batch of cells, such as from a working cell bank (WCB).

The cells may be grown in adherent mode in the bioreactor. In order to increase adherent surface, the bioreactor may contain a carrier onto which expanded cells can anchor. Such carrier may be either floating or fixed in the bioreactor. Illustrative bioreactors include fixed bed bioreactor. Illustrative carriers include, without limitation, fiber carriers, microcarriers (e.g. beads) and macrocarriers, in particular fiber carriers such as packed-fiber carrier. Such carriers can be made, for example, using polyethylene terephthalate, polystyrene, polyester, polypropylene, DEAE-dextran, collagen, glass, alginate or acrylamide. Suitable bioreactors which may be used include those containing bead-type micro-carriers (e.g., Cytodex® brand beads, commercially available from Cytiva) and matrix type carriers (e.g., Fibra-cell™ brand disks, commercially available from Eppendorf Corp.). As an illustration, the bioreactor may comprise a polyester packed-fiber carrier such as that used in the iCELLis® 500 or iCELLis® 500+ bioreactors, (commercially available from Advanced Technology Materials Inc. (Brussels, Belgium) and Cytiva). The bioreactor may comprise a carrier providing a surface area for cell growth comprised between about 66 m² and about 500 m², in particular between 100 m² and 500 m². The bioreactor may for example comprise a polyester packed-fiber carrier such as that used in the iCELLis® 500 or iCELLis® 500+ bioreactors providing a surface area for cell growth comprised between about 66 m² and about 500 m².

The medium used for growth into the bioreactor may comprise a defined medium, containing components whose exact concentration is known, such as a basal medium basal medium (e.g. DMEM, F12 or RPMI medium). The medium further contains factors suitable to promote cell adherence to the bioreactor or carrier contained therein. Such factors promoting adherence include, without limitation, cell adhesion factors, such as components of the extracellular matrix (e.g. fibronectin, collagen, laminin, calcium ions, or proteoglycans or non-proteoglycan polysaccharides of the extracellular matrix) and animal-derived serums, such as mammal derived serum, in particular fetal bovine serum (FBS). In particular, the medium may comprise animal-derived serum, in particular FBS. Such factors suitable to promote cell adherence may be added to the culture medium just before, during or after the inoculation of the suspension cells into the bioreactor. Suitable media include, without limitation, DMEM, F12 or RPMI medium, in particular DMEM, supplemented with FBS, for example FBS at a concentration between about 2 and about 20% v/v of the medium, in particular between about 5 and about 15%, e.g., about 9%. The medium may further comprise other components such as L-glutamine (for example at a concentration of about 0.5-about 5 mM, such as about 2 mM) or L-alanyl-L-glutamine (for example at a concentration of about 0.5-about 5 mM, such as about 2 mM).

Inoculation of the bioreactor with an effective quantity of cells is performed, with said quantity varying depending on the bioreactor to be used. In certain variants, inoculation may be performed to achieve a cell density of about 1000-about 40,000 cells/cm², in particular of about 2,000-about 20,000 cells/cm², more particularly of about 6,000 to about 15,000 cells/cm², more particularly of about 7,000-about 12,000 cells/cm², in particular of about 8,000-about 10,000 cells/cm². By way of an example, a bioreactor providing a surface area of about 100 m², such as the iCELLis® 500+/100 m² bioreactor, is inoculated in a particular embodiment with between about 1.0×10⁹ and about 1.0×10¹¹ cells, in particular between about 0.6 and about 1.5×10¹⁰ cells, more particularly between about 0.8 and about 1.0×10¹⁰ cells. By way of another example, a bioreactor can be used that provides a surface area of about 500 m², such as the iCELLis® 500+/500 m² bioreactor, in which in a particular embodiment, between about 5.0×10⁹ and about 1.0×10¹¹ cells, in particular between about 2.0 and about 7.0×10¹⁰ cells, more particularly between about 3.0 and about 6.0×10¹⁰ cells, in particular between about 4.0 and about 5.0×10¹⁰ cells are inoculated into the bioreactor.

Culture volume may vary to a large extent, depending on the cells and type of bioreactor used for the production of the recombinant adenovirus vector. For example, when the cells are HEK293 cells or cells derived therefrom, the bioreactor can comprise (a) a polyester packed-fiber carrier providing a surface area for cell growth comprising between about 66 m² and about 500 m², and (b) from about 40 to about 100 L of culture medium, in particular from about 50 to about 80 L, such as from about 60 to about 70 L of culture medium, and in particular about 65 L of culture medium.

Cells may then be expanded for a period of time allowing reaching a cell density suitable for production of clinical or commercial batches of recombinant adenovirus vectors. For example, cells may be expanded in the bioreactor for about 70 to about 150 hours before infection.

### Infection

In step c), inoculated cells are infected with the recombinant adenovirus vector to be propagated. In certain embodiments, infection of the cells may be performed at a multiplicity of infection (MOI) of about 50 to about 400 vp/cell, such as from about 100 to about 200 vp/cell. In certain other embodiments, the MOI is of about 3 to about 50 infectious particles/cell, such as from about 6 to about 25 infectious particles/cell.

One need to determine the cell count in the bioreactor in order to determine the number of viral particles required to achieve the target MOI. However, counting of cells may not always be possible, depending on the type of bioreactor used, as is the case with a packed-fiber carrier made of polyester providing a surface area for adherent cell growth. In that case, the number of cells in the bioreactor can be theoretically determined based on the seeding density, expansion time between inoculation and infection, and doubling time of the cells used in the production. Determination of these parameters is routine practice for those skilled in the art. For example, with cells with a doubling time of about 24 hours, and inoculation at a cell density of about 10,000 cells/cm², after about 100 hours cultivation the cell density would be of about 180,000 cells/cm². As a further illustration, with cells with a doubling time of about 29 hours, and inoculation at a cell density of about 10,000 cells/cm², after about 110 hours cultivation the cell density would be of about 140,000 cells/cm².

In an illustrative example, the cells, such as HEK293 cells or HEK293 related cells, more particularly HEK293 cells, are infected at a cell density of about 80,000 to about 150,000 cells/cm².

Duration of infection may vary from about 30 to about 80 hours.

The cell culture may be carried out in batch or in perfusion mode. In certain implementations, the cell culture is carried out in perfusion mode. In such variant, perfusion may be stopped just before infection, and the required volume of virus is added to the bioreactor to infect the expanded cells. Perfusion may be restarted about 30 to about 120 minutes after the start of the infection of the cells. Moreover, approximately about 15 to about 50 hours post-infection, the perfusion media may be changed to serum-free media. This allows reducing the concentration of residual FBS at harvest. Perfusion may be continued until such harvest.

### Cell Lysis, Vector Harvest and Filtration

The formation of recombinant adenovirus vectors takes place inside the cells. Thus, release of such vectors may be carried out using a lysis step. Cell lysis may be carried out by any conventional physical or chemical means. In particular, lysis may be carried out inside the bioreactor by adding at least one detergent into to the bioreactor, such as a non-ionic surfactant. Examples of non-ionic surfactants that can be used as detergents include polysorbate, in particular polysorbate 20, or Triton-X. In particular, the detergent may be polysorbate, in particular polysorbate 20. In particular, lysis may be carried out with a lysis buffer comprising about 10% m/v polysorbate 20, such as about 10% m/v polysorbate. The spent media in the bioreactor may be either kept or discarded, preferably discarded, before addition of the lysis buffer, and replaced by fresh serum-free medium, such as serum-free DMEM medium. Lysis is then continued for a sufficient time, such as for about 1 to about 3 hours, in particular for about 2 hours±about 10 minutes.

Endonuclease treatment may be carried out to digest host cell nucleic acid molecules. The endonuclease treatment may be carried out any enzyme able to degrade DNA and/or RNA, preferably both. Such endonucleases include, without limitation, an endonuclease from Serratia marcescens, in particular a genetically engineered endonuclease from Serratia marcescens such as Benzonase®. Endonuclease treatment may be implemented as a two-step endonuclease treatment. Accordingly, a first endonuclease (e.g. Benzonase®) treatment is carried out before addition of the lysis buffer, and a second endonuclease (e.g. Benzonase®) treatment is carried out after addition of the lysis buffer. For example, in an illustrative, non-limiting example, after replacement of spent media with fresh serum-free media, a first Benzonase® aliquot of approximately about 60 to about 100 U/mL, such as about 74 U/mL, is added to the bioreactor and incubated for about 25 to about 40 minutes, in particular for about 30 to about 35 minutes. Lysis buffer is then added and lysis is continued for the required time period. Then, a second Benzonase® aliquot of approximately about 300 to about 450 U/mL, such as, for example, about 369 U/mL, may be added to the bioreactor to digest host DNA for an additional about 25 to about 40 minutes, in particular for an addition about 30 to about 35 minutes. Benzonase may then be inactivated with a high concentration salt buffer, such as a buffer comprising about 3,500 to about 5,000 mM NaCl.

The recombinant adenovirus vectors can then be harvested by collecting the lysed material, thereby providing a main harvest material. In certain cases, the bioreactor may then further be rinsed with conditioning buffer to recover as many virus particles from the bioreactor as possible. Both the main harvest material and rinse may then be pooled. The main harvest, optionally pooled with the rinse, may be referred to as a bulk harvest.

Step e) of filtering said bulk harvest is then implemented to obtain a filtered bulk harvest. Such filtration may be carried out through an about 0.2 μm filter, such as about a 0.5/0.2 filter. The filter may further be flushed with conditioning buffer. The resulting product is referred to as a filtered bulk harvest.

Of course, it should be understood that such filtered bulk harvest may be produced using other methods for the production of a recombinant adenovirus vector than the method described above.

### 4. Method for the Purification of Recombinant Adenovirus Vectors

The present disclosure relates to a method for the purification of a recombinant adenovirus vector, comprising the steps of submitting a preparation of recombinant adenovirus vectors to two-step anion exchange chromatography, and recovering the recombinant adenovirus vectors from the two-step anion exchange chromatography elute.

The preparation of recombinant adenovirus vectors applied to the two-step anion exchange chromatography may be a bulk harvest of recombinant adenovirus vectors, a filtered bulk harvest of recombinant adenovirus vectors, recombinant adenovirus vectors that have been submitted to preliminary purification steps, or recombinant adenovirus vectors that have been submitted to one or more conditioning steps.

In a particular embodiment, the preparation of recombinant adenovirus vectors applied to the two-step anion exchange chromatography is a bulk harvest or a filtered bulk harvest which has been submitted to one or more concentration, diafiltration or concentration and diafiltration steps.

Harvested recombinant adenovirus vectors, such as a bulk harvest or a filtered bulk harvest of recombinant adenovirus vectors, may be submitted to a concentration and diafiltration step, in particular to a tangential flow filtration (TFF) step. This step may be implemented to concentrate and condition the recombinant adenovirus vectors prior to the subsequent chromatography step. Different types of membranes and filter formats are available for TFF. In a particular embodiment, a membrane or filter with a molecular weight cuf-off (MWCO) comprised between 30 and 300 kDa, in particular between 100 and 300 kDa, more specifically of 300 kDa, is implemented. In yet another embodiment, a TFF membrane or filter made of polyethersulfone (PES), modified PES or regenerated cellulose, in particular PES, may be used. In a particular embodiment, the TFF membrane of filter has a MWCO of 300 kDa and is made of PES. The TFF may be used to concentrate the recombinant adenovirus vector, and diafiltering the concentrated vector. The concentration step may be carried out to concentrate the recombinant adenovirus vector at least about 3 times, such as at least about 4 times, in particular about 5 times or more, depending on the target volume, as compared to the volume of the harvest. Diafiltration may be implemented to condition the recombinant adenovirus vector. Diavolume may be of at least about 2×, such as at least about 3×, in particular at least about 4×, at least about 5×, or at least about 6×. In a particular embodiment, the diavolume is of about 7×. After diafiltration, the product may be further concentrated before the subsequent chromatography step. Concentration after diafiltration may in particular be implemented to concentrate two times the diafiltered product. After the first tangential flow filtration step, the resulting product may be filtered, in particular through about a 0.5/0.2 μm filter. The product resulting from the TFF step, optionally further filtered, is an example of a preparation of recombinant adenovirus vectors that can be submitted to the two-step anion exchange chromatography as provided below.

According to the disclosure, a preparation of recombinant adenovirus vectors is submitted to a two-step anion exchange chromatography. This purification step comprises two consecutive anion exchange chromatography steps. Indeed, the present inventors have shown that surprisingly, given the non-orthogonal nature of two anion exchange chromatography steps, the two-step anion exchange chromatography described herein greatly improves the purity of the recombinant adenoviral vector and its recovery.

Before the two-step anion exchange chromatography, the preparation of recombinant adenovirus vectors may first be diluted in a dilution buffer devoid of salt, in particular devoid of NaCl, to reach a target conductivity suitable to preferentially bind the adenovirus vector to the anion exchanger while protein impurities with high pI and small digested DNA fragments do not bind. Such target conductivity may be of 30-36 mS/cm. The dilution buffer can optionally comprise a detergent such as a non-ionic surfactant, to prevent aggregation. An illustrative suitable dilution buffer comprises about 1% m/v polysorbate 20, at a pH of about 7.5.

In a particular embodiment, strong anion exchange chromatography is performed. Strong anion exchange chromatography may be performed on a strong anion exchange resin, monolith or membrane. More specifically, anion exchange chromatography columns are available with matrices having either strong or weak functional groups and are well known in the art. As is well known in the art, strong anion exchange resins contain quaternary ammonium functional groups (e.g., quaternary ammonium groups (R4N+) attached to a polymeric backbone). These groups are strong bases, allowing the resin to exchange anions in a wide pH range (e.g., a pH of 0-14). There are two types of quaternary ammonium functional groups: Type I resins contain trialkyl ammonium chloride or hydroxide, and Type II resins contain dialkyl 2-hydroxyethyl ammonium chloride or hydroxide. Unlike weak anion exchange resins (e.g., diethylaminoethyl or DEAE), strong anion exchange resins remain ionized under alkaline conditions, enabling consistent binding performance. Strong anion exchange resins can have a particle size of about 50 to 90 μm and exhibit a high dynamic binding capacity (DBC) (e.g., >75 mg/mL for proteins, >140 mg/mL for plasmid DNA) which allows efficient capture of impurities like host cell proteins (HCPs) and viruses. Strong anion exchange resins are available in several forms, including beads with dense internal structures (gel resins), porous structures (macroporous resins), and membranes. Examples of strong anion exchange membranes include, without limitation, SartobindQ membranes. Illustrative commercially available strong anion exchange chromatography beads include, without limitation, an Eshmuno Q resin, a CaptoQ resin, a CaptoQ ImpRes resin, or a Source 15Q resin. Examples of strong anion exchange matrix is quaternary ammonium, and is usually designated Q. Examples of weak anion exchange matrix is diethylaminoethyl, or DEAE.

In a particular embodiment, two-step anion exchange chromatography comprises a first anion exchange chromatography on a strong anion exchange membrane and a second anion exchange chromatography on a strong anion exchange resin. Illustrative commercially available strong anion exchange membranes include, without limitation, SartobindQ membranes. Illustrative commercially available strong anion exchange chromatography resins include, without limitation, the Eshmuno Q resin, CaptoQ resin, the CaptoQ ImpRes resin and the Source 15Q resin.

The optionally diluted preparation of recombinant adenovirus vectors is loaded onto the first anion exchanger. In a particular embodiment, the first anion exchanger is a membrane or resin anion exchanger, in particular a membrane exchanger such as the SartobindQ membrane. After loading, the anion exchanger may be washed with a low salt buffer, such as a buffer A comprising about 200 to about 400 mM NaCl. The low salt buffer may also comprise a detergent, such as polysorbate, in particular polysorbate 20, such as polysorbate 20 at about 0.01 to about 0.5% m/v, in particular at about 0.1% m/v. The recombinant adenovirus vector may then be separated from bound impurities and eluted using a linear salt gradient, by addition of a high salt buffer, such as a buffer B comprising about 1,000-about 2,000 mM NaCl. The high salt buffer may also comprise a detergent, such as polysorbate, in particular polysorbate 20, such as polysorbate 20, in particular at about 0.01 to about 0.5% m/v, in particular at about 0.1% m/v. Such linear gradient may be, for example, from about 0% to about 50% of buffer B added to buffer A. Virus peak collection can be monitored by UV absorbance (A280). The recombinant adenovirus vector fraction is then collected for loading on the second anion exchanger.

In a particular embodiment, after the first anion exchange chromatography step, a detergent, such as polysorbate, in particular polysorbate 20, such as polysorbate 20 at a concentration of about 0.1 to about 1.5% m/v, in particular at a concentration of about 1% m/v, may be added to the first chromatography product. For example, in a particular embodiment, when polysorbate is used, the polysorbate concentration can be adjusted by addition of an incubation buffer comprising about 600 mM NaCl and about 20% m/v polysorbate, such as polysorbate 20.

The second anion exchange chromatography step can then be performed on the collected recombinant adenovirus vector fraction. Before the second chromatography purification, the salt concentration of the first chromatography product can be decreased (diluted) to about 30-36 mS/cm conductivity using a dilution buffer devoid of salt, in particular of NaCl. The dilution buffer may comprise a detergent at the concentration of the high salt buffer and/or low salt buffer, for example polysorbate, such as polysorbate 20, at a concentration of about 0.1 to about 1.5% m/v, in particular at a concentration of about 1% m/v. In a particular embodiment, the second anion exchange chromatography step is a strong anion exchange chromatography. In yet another embodiment, the second anion exchange chromatography step is performed on a strong anion exchange chromatography resin or membrane, in particular on a strong anion exchange chromatography resin. Such resin may be packed in a column. The recombinant adenovirus vector fraction is loaded onto the second anion exchanger, such as an anion exchange resin as discussed above. In a particular embodiment, the anion exchanger may be washed with a low salt buffer, such as a buffer A as described above, and eluted using a linear salt gradient. In linear gradient, the salt concentration of the low salt buffer is increased with a high salt buffer, such as the buffer B described above, from about 300 mM to about 900 mM NaCl to elute the recombinant adenovirus vector. The virus peak collection is defined based on UV absorbance (A280). In a particular embodiment, recombinant adenovirus vectors elute at a conductivity more than about 48 mS/cm.

The virus peak collection can be ended when A280 reaches about 13 to about 15% of the peak max.

After the chromatographic purification steps, the eluate from the second anion exchange chromatography step may be further processed.

In a particular embodiment, the eluate of the second anion exchange chromatography is submitted to TFF. The aim of this TFF step is to desalt the elution product, to exchange buffers to the final formulation buffer of drug substance, and to adjust the concentration of the recombinant adenovirus vector. In a particular embodiment, the TFF step carried out on the second anion exchange chromatography eluate is implemented with a membrane or filter with a molecular weight cut-off (MWCO) of 30 to 300 kDa, in particular of 100 to 300 kDa, in particular 300 kDa. In yet another embodiment, a TFF membrane or filter made of polyethersulfone (PES), modified PES or regenerated cellulose, in particular PES, may be used. In a particular embodiment, the TFF membrane of filter has a MWCO of 300 kDa and is made of PES. The product may be concentrated before diafiltration. Diafiltration may be performed to condition the recombinant adenovirus vector. The conditioning buffer is selected as suitable to condition the produced recombinant adenovirus vector. For example, the conditioning buffer may comprise 10.9 mM Sodium Phosphate, 14 mM Tris base, 2 mM MgCl₂, 2% m/v Sucrose, 10% m/v Glycerol, at pH 7.9. The diavolume may be of at least about 10×, such as at least about 12×, in particular at least about 13×, at least about 14×, or at least about 15×. In a particular embodiment, the diavolume is about 15×. After diafiltration, the product may be further concentrated to target concentration based on total virus particle count. After the this TFF step, the resulting product may be filtered, in particular through about a 0.2 μm filter.

The resulting product is also referred to as a drug substance. After purification, said drug substance may be stored frozen at temperature equal or lower to −60° C. until use for formulation into the drug product intended to be administered to a subject in need thereof.

### 5. Method for the Manufacture of a Ready-to-Use Drug Product

Another aspect described herein relates to a method for the manufacture of an injectable ready-to-use (RTU) drug product comprising a recombinant adenovirus vector, said RTU drug product being suitable for human use in gene therapy. In a particular embodiment, the drug product comprises a rAd5 vector. In another particular embodiment, the rAd5 vector is for instillation into the bladder of a subject in need thereof. In yet another embodiment, the rAd5 vector encodes a human Type 1 or Type 2 interferon, in particular human Type 1 interferon, such as human interferon α or β, in particular human interferon α, more particularly interferon α2, even more particularly human interferon α2b. In yet another embodiment, the drug product is nadofaragene firadenovec, which is a gene therapy vector that carries the human interferon α2b cDNA in an expression cassette in place of the E1a and E1b regions at the 5′ end of the adenoviral genome, further comprising Syn3/NODA, [N-(3-cholamidopropyl)-N-(3-lactobionamidopropyl)]-cholamide, an excipient included in the formulation to facilitate uptake of the vector into bladder epithelial tissue and expression of interferon α2b.

It has been observed that nadofaragene firadenovec is sensitive to heat and terminal sterilization can therefore not be applied. The RTU drug product suspension is thus manufactured as disclosed herein through aseptic processing and sterilized by filtration through a sterilizing grade filter.

The method for the manufacture of the RTU drug product disclosed herein comprises the steps of (i) mixing a drug substance with a Syn3/NODA solution and a final formulation buffer (FFB) solution, and (ii) filtering the resulting drug product with a sterilizing filter. In a particular embodiment of the method for the manufacture of the RTU drug product, first a FFB is added to the drug substance, then a Syn3/NODA solution is added. A mixing step can then be implemented before filtration. This specific sequence of steps was shown to be optimal to streamline the formulation process.

As a result of the manufacturing method disclosed herein, the pH of the drug product is maintained at about 7.8, more specifically at pH 7.8, to optimally stabilize the drug product. This pH is maintained using sodium dihydrogen phosphate dihydrate and tromethamine.

Sucrose, magnesium chloride hexahydrate and glycerol are also included in the formulation to buffer and stabilize the product. The inventors have shown that the product remains stable for a suitable time period between thawing and administration. The present disclosure thus provides a method for the manufacture of a drug product which is convenient to handle, because of its stability in the frozen state, but also for a suitable time period after thawing.

The drug substance, i.e. a recombinant adenovirus vector, may be a vector purified according to the method of purification described above. If the drug substance is in a frozen state before manufacture of the drug product, the drug substance is first thawed before formulation with Syn3/NODA and the FFB.

In some embodiments, a step of preparing a Syn3/NODA solution is implemented. In a particular embodiment, the Syn3/NODA solution is an aqueous solution comprising Syn3/NODA and water for injection (WFI). The Syn3/NODA solution may further comprise one more other components, such as:
a cyclodextrin, in particular hydroxypropyl-beta dextrin;
citric acid, such as citric acid monohydrate;
citrate, such as trisodium citrate dihydrate; and
a detergent such as polysorbate, in particular polysorbate 80.

In a particular embodiment, the following Syn3/NODA solution is prepared before mixing:

| | | |
|---|---|---|
| Hydroxypropyl-beta-cyclodextrin | 49 | mg/mL |
| Syn3/NODA | 5.8 | mg/mL |
| Citric acid monohydrate | 0.08 | mg/mL |
| Trisodium citrate dihydrate | 0.25 | mg/mL |
| Polysorbate 80 | 2.9 | mg/mL |

| | |
|---|---|
| WFI | q.s. |

In a particular embodiment, the Syn3/NODA solution is prepared by weighing and adding each ingredient into a mixing vessel, such as a mixer bag, in particular a single-use mixer bag. In a particular embodiment, the ingredients are added sequentially until each is completely dissolved and/or mixed. In a particular embodiment, preparation of the Syn3/NODA solution comprises the steps of:
adding water for injection (WFI) to a mixing vessel, in particular to approximately 11% of final weight;
adding hydroxypropyl-beta-cyclodextrin to the vessel, and stirring until completely dissolved;
adding Syn3/NODA to the vessel and stirring until completely dissolved;
adding citric acid monohydrate, trisodium citrate dihydrate, and WFI to a flask, such as a single-use flask, to prepare a buffer solution, and the content is dissolved;
adding the buffer solution to the mixer vessel and stir until completely mixed;
adding polysorbate 80 and WFI to a flask, such as a single use flask, mixing the content of the flask, adding the content of the flask to the mixer vessel, and stir until completely mixed; and
adding WFI to final weight and stir until completely mixed.

In a particular embodiment, the Syn3/NODA solution is filtered for bioburden reduction through a sterilising grade filter, such as a 0.2 μm membrane filter, in particular a sterilising grade 0.2 μm membrane filter, such as a polyethersulfone 0.2 μm membrane filter. The Syn3/NODA solution may be stored at about 2 to about 8° C.

In some embodiments, a step of preparing a FFB solution is implemented. In a particular embodiment, the FFB solution is an aqueous solution comprising one more components such as:
sodium phosphate, such as sodium dihydrogen phosphate dihydrate;
tromethamine;
sucrose;
magnesium chloride, such as magnesium chloride hexahydrate; and
glycerol.

In a further particular embodiment, the following final formulation buffer is prepared before mixing:

| | | |
|---|---|---|
| Sodium dihydrogen phosphate dihydrate | 1.7 | mg/mL |
| Tromethamine | 1.7 | mg/mL |
| Sucrose | 20 | mg/mL |
| Magnesium chloride hexahydrate | 0.41 | mg/mL |
| Glycerol | 100 | mg/mL |

| | |
|---|---|
| WFI | q.s. |

In a particular embodiment, the FFB solution is prepared by weighing and adding each ingredient into a mixing vessel, such as a mixer bag, in particular a single-use mixer bag. In a particular embodiment, the ingredients are added sequentially until each is completely dissolved and/or mixed. In a particular embodiment, preparation of the FFB solution comprises the steps of:
adding WFI to a mixing vessel, in particular to approximately 88% of final weight;
adding sodium dihydrogen phosphate dihydrate to the vessel, and stirring until completely dissolved;
adding tromethamine to the vessel and stirring until completely dissolved;
adding sucrose to the mixer vessel and stir until completely mixed;
adding magnesium chloride hexahydrate to the mixer vessel and stir until completely mixed; and
adding glycerol to the mixer vessel and stir until completely mixed.

In a particular embodiment, the FFB solution is filtered for bioburden reduction through a sterilising grade filter, such as a 0.2 μm membrane filter, in particular a sterilising grade polyethersulfone 0.2 μm membrane filter. The FFB solution may be stored at about 2 to about 8° C.

In a particular embodiment, the method for the manufacture of the RTU drug product disclosed herein comprises a step of adding the drug substance to a mixer vessel, such as a single-use bag, adding to the mixer vessel the FFB solution, and then adding the Syn3/NODA solution, and then mixing the content of the mixer vessel. In a particular embodiment, the content of the mixer vessel is gently mixed until homogenous. Suitable gentle mixing include, without limitation, placing the mixer vessel on a rocker.

In a particular embodiment, the manufactured drug product comprises:
about 3×10¹¹ vp/mL nadofaragene firadenovec,
about 0.95 mg/mL Syn3,
about 0.01 mg/mL citric acid monohydrate,
about 0.04 mg/mL Tri-sodium citrate dihydrate,
about 0.48 mg/mL polysorbate 80 (Tween 80),
about 7.9 mg/mL hydroxypropyl-beta-cyclodextrin,
about 1.4 mg/mL sodium dihydrogen phosphate dihydrate,
about 1.4 mg/mL tromethamine,
about 17 mg/mL sucrose,
about 0.34 mg/mL magnesium chloride hexahydrate,
about 84 mg/mL glycerol, and
Water for Injection (q.s.).

The method disclosed herein further comprises filtering the drug product with a sterilising filter. In some embodiments, the sterilising filter is a sterilising 0.2 μm filter, in particular a sterilising 0.2 μm membrane filter. In yet another embodiment, the filter is a polyethersulfone filter. In a further embodiment, the filter is pre-rinsed with a pre-rinsing solution before filtration of the drug product. Such pre-rinsing solution may be prepared by mixing a Syn3/NODA solution with a FFB solution, such as the solutions described above, in proportions equivalent to that present in the final drug product. Thus, in some embodiments, a step of preparing a blank solution for use in pre-rinsing a sterilizing filter is implemented.

The drug product may then be filled in one or more vials, packaged, and stored frozen at a temperature equal or lower to −60° C.

### EXAMPLES

### Example 1. Thawing and Expansion of HEK293 Cells from Working Cell Bank (WCB)

The purpose of this process stage is to expand HEK293 cells from a WCB vial to produce enough cells for the inoculation of an adherent single-use bioreactor comprising an iCELLis 500+ carrier.

A vial of WCB were removed from liquid nitrogen and cells thawed by partial immersion of the vial in a water bath at +37° C. Cells were transferred to a 75 cm² cell culture flask and incubated in +37° C. and 5% CO₂ for 3-4 days.

When expanding the cells in T-flasks and roller bottles, the cell number and viability was determined at each passage. Cell suspension was diluted with fresh cell culture medium. In a first expansion, cells were seeded to 1 to 2×75 cm² flasks and incubated at +37° C. and 5% CO₂ for 3-4 days. In the second expansion cells were seeded to 2 to 3×75 cm² flasks or one 850 cm² roller bottle, depending on the total number of cells and the total cultivation volume. Cells were cultivated at +37° C. and 5% CO₂ for 3-4 days.

In the third expansion, cells were expanded and transferred to 1 roller bottle within the volume. Cells were expanded further until the amount of 10 roller bottles (850 cm²) was achieved. Cells were grown in incubators (+37° C., 5% CO₂), on a roller device.

This cultivation process resulted in 10 roller bottles containing cells in a volume of 1.5 L and a total of about 1.0×10⁹ to about 1.0×10¹¹ cells.

Cells were transferred from roller bottles into a perfusion bioreactor for further expansion. The cultivation volume was increased from 5 L to 10 L (final working volume) within following two days. Approximately 24 h after the 10 L working volume was reached, perfusion was started to enhance cell proliferation to reach the target cell density of 0.5×10⁶ to 0.5×10⁷ cells/mL.

Perfusion was continued until the target cell number is reached.

### Example 2. Adherent Cell Expansion and Infection in iCELLis 500+/500 m², Harvest and Bulk Harvest Filtration

Cells from the previous expansion step were diluted into Ex-Cell medium to form an 8 L to 10 L aliquot that was inoculated into the iCELLis 500+/500 m² bioreactor to achieve a cell density of 8000-10,000 cells/cm². Cells were inoculated into the iCELLis 500+/500 m² bioreactor containing 60 L of media (DMEM-9% FBS-2 mM L-GlutaMAX).

Cell proliferation was enhanced by perfusion, started on the second day of cell proliferation in the iCELLis 500+/500 m².

Before the infection, the number of cells was assessed in order to calculate the correct MOI. It is not possible to count cells from the adherent iCELLis 500+/500 m² bioreactor. Therefore, for each batch, the number of cells in the bioreactor was determined theoretically based on the seeding density, expansion time between inoculation and infection, and doubling time of the cells characteristic to HEK293 WCB. Theoretically, when cells have been inoculated at a cell density of 10000 cells/cm², after 110 h cultivation the cell density would be 140000 cells/cm².

A sufficient amount of a secondary Working Virus Seed Stock (sWVSS) aliquots was thawed in a water bath set at +37° C. Infection of HEK293 cells was performed using a MOI 150-170 vp/cell, 9-21 infectious particles/cell. Just before the infection, the perfusion was stopped, and the required volume of virus was added to the iCELLis 500+/500 m² system to infect the expanded HEK293 cells. Duration of infection was of 50-60 hours.

Approximately 60 minutes after cells have been infected, the perfusion was restarted. Approximately 30 h post infection, the perfusion media was changed to serum-free media, reducing the concentration of residual FBS at harvest. Perfusion was continued until harvest.

Spent media in the bioreactor was discarded. The bioreactor was refilled with 60 L of fresh DMEM without additives and allowed to equilibrate for 0-30 minutes until it was in the target temperature range (≥29.5° C.). A primary Benzonase treatment aliquot of approximately 74 U/mL was added to the bioreactor and incubated for 30-35 minutes with temperature control and stirring on. The purpose of the primary Benzonase treatment was to start DNA digestion as soon as the host cell DNA begins to be released from the cells during the lysis step.

Cell lysis was performed with lysis buffer containing 10% m/v polysorbate 20. Lysis was continued for 2 hours+10 minutes. A secondary Benzonase treatment of approximately 369 U/mL was then added to digest host cell DNA for a further 30-35 minutes prior to inactivation of the Benzonase with a high concentration salt buffer (incubation time 15-20 minutes at 30-37° C. with constant stirring). After incubation, the content of the bioreactor was collected into a harvest bag. A total of approximately 87 L was recovered. The bioreactor was further rinsed with 120 L of conditioning buffer to recover as many virus particles from the bioreactor bed as possible. The rinse was pooled with the main harvest material. This pooled harvest material was referred to as “Bulk Harvest” and was further filtered through a 0.5/0.2 μm filter, after which the filters are flushed with total of 20 L of conditioning buffer to obtain a “filtered bulk harvest”.

### Example 3. Purification Method Comprising a Two-Step Anion Exchange Chromatography

A TFF system was used for concentration and conditioning of the Filtered Bulk Harvest material produced in the iCELLis 500+/500 m² system.

First, TFF systems with a MWCO of 300 kDa and 1000 kDa were compared. The 300 kDa MWCO cassette good DNA and protein clearance and suitable vector recovery. With respect to the 1000 kDa MWCO cassette, although DNA and protein clearance was very high, virus particle amount was drastically decreased during the concentration and diafiltration steps. Therefore, 300 kDa MWCO TFF cassettes were retained for further processing.

Product was concentrated prior diafiltration and filtrate flow meter is used to determine when 7× diafiltration has been completed.

At the end of the process the product was recovered from the system (product intermediate ‘TFF1 product’), filtered through a 0.5/0.2 μm filter (product intermediate ‘Filtered TFF1 product’) and held at ambient temperature overnight prior to chromatography purification.

Concentrated and conditioned feed material from the first tangential flow filtration step (TFF1) contained impurities originating from the host cells (e.g., host cell DNA and host cell proteins), concentrated polysorbate 20 and possible cell culture medium derived impurities. The Filtered TFF1 product was diluted with Dilution buffer to a target conductivity of ˜30 mS/cm. The first chromatography step used strong anion exchange Sartobind Q membrane cassettes. The product was bound to the membrane during loading, whilst a portion of unwanted proteins and residues were flushed to waste at low conductivity. Membrane was washed with A-buffer (low salt buffer) and the virus was separated from bound impurities by elution with a linear salt gradient. Linear gradient from 0% to 50% of B-buffer (high salt buffer) was added to A-Buffer in 40 membrane volumes.

The virus peak collection was determined by UV absorbance (A280). The virus eluted at a conductivity >˜43 mS/cm. The virus peak collection was ended when A280 reaches 10-15% of the peak max. The virus fraction (‘Chromo 1 product’) was collected.

The Chromo 1 product was first directly submitted to a TFF step as described below. However, it was observed that the permeate flux decreased during processing, scale of recombinant adenovirus vector was low, and host cell protein impurities were not sufficiently eliminated.

Therefore, it was concluded that further purification should be conducted. It was decided to apply the Chromo 1 product to a second chromatography step to further purify Chromo 1 product by removing residual host cell protein impurities.

Different anion exchangers were screened for the second chromatography step. These included a strong anion exchange membrane (second Sartobind Q membrane) and different weak (Fractogel DEAE) and strong (Source 15Q, Capto Q, Capto Q Impress, Eshmuno Q) anion exchange resins packed in columns.

Immediately after the Chromo 1 purification run, the polysorbate concentration in the Chromo 1 product was increased to 1% by adding incubation buffer and the product was incubated at room temperature. Before the second chromatography purification, the salt concentration of the Chromo 1 product was decreased (diluted) to ˜30 mS/cm conductivity using dilution buffer.

The diluted virus fraction was submitted to the different anion exchangers.

For the second membrane anion exchange chromatography, different runs were performed. One with and one without preliminary filtration. Membrane was washed with A-buffer (low salt buffer) and the virus was separated from bound impurities by elution with a shallow salt gradient from 0% to 50% of B-buffer (high salt buffer) was added to A-Buffer in 50 membrane volumes and then 100% of B-buffer in 4 membrane volumes.

For the assays with resins, the columns were washed with A-buffer and eluted using a linear salt gradient. In linear gradient, the salt concentration of A-buffer was increased with B-buffer (linear gradient from 0% to 50% of B-buffer) to elute the product. The virus peak collection was defined based on UV absorbance (A280). The virus eluted at a conductivity >˜48 mS/cm. The virus peak collection was ended when A280 reaches 13-15% of the peak max.

Protein purity, recovery and processing time were the three factors considered for evaluation of the different exchangers. Membrane chromatography as a second anion exchange step was left out as it was clear from an SDS-PAGE analysis that impurity clearance with such membrane was not high enough. Moreover, weak anion exchange resin suffered from low purity and also from long processing time. On the other hand, strong anion exchange resins resulted in the most optimal second anion exchange step, considering the three evaluation factors retained. Thus, it has been observed that first, although not orthogonal, a second anion exchange chromatography step allows to further purify the eluate of a first anion exchange chromatography step and, secondly, that the best performing exchangers as the second anion exchange step is a strong anion exchange resin.

After the strong anion exchange resin chromatographic purification step, the product ‘Chromo 2 product’ was transferred to the second tangential flow filtration for formulation of the recombinant adenovirus vector.

The aim of the second tangential flow filtration step (TFF2) was to de-salt the chromatography elution components (salts), change to the final formulation buffer of Drug Substance and to concentrate for adjusting the final recombinant adenovirus vector concentration. In this step, the “Chromo 2 product” was formulated in Final Formulation Buffer by TFF through three 0.5 m² 300 kDa cassettes (1.5 m² total membrane area) and the product concentration was adjusted to target concentration based on “Chromo 2 product” total virus particle count. Final formulation buffer comprised 10.9 mM Sodium Phosphate, 14 mM Tris base, 2 mM MgCl₂, 2% Sucrose, 10% Glycerol, at pH 7.9.

Product was concentrated prior diafiltration and filtrate flow meter is used to determine when 15× diafiltration has been completed. The product concentration was adjusted to target concentration based on “Chromo 2 product” total virus particle count.

On completion of TFF diafiltration the product was recovered from the equipment and called “TFF2 product”. TFF2 product was further filtered through a 0.2 μm PES filter. The filtered product was stored frozen below −60° C.

### Example 4. Formulation of a Ready-to-Use Drug Product

This example reports the preparation of a ready-to-use recombinant adenovirus vector formulation.

Two solutions were prepared before the drug product suspension was formulated: a Syn3/NODA solution and a final formulation buffer (FFB) solution.

| Syn3NODA solution | Hydroxypropyl-beta-cyclodextrin | 49 | mg/mL |
|---|---|---|---|
| | Syn3NODA | 5.8 | mg/mL |
| | Citric acid monohydrate Trisodium citrate | 0.08 | mg/mL |
| | dihydrate Polysorbate 80 | 0.25 | mg/mL |
| | WFI | 2.9 | mg/mL |
| | | q.s. | |
| FFB solution | Sodium dihydrogen phosphate dihydrate | 1.7 | mg/mL |
| | Tromethamine | 1.7 | mg/mL |
| | Sucrose | 20 | mg/mL |
| | Magnesium chloride hexahydrate Glycerol | 0.41 | mg/mL |
| | WFI | 100 | mg/mL |

q.s.

A blank solution was also prepared for pre-rinse of the filter in the last sterile filtration step. The blank solution was a mixture of Syn3/NODA solution (approximately 16%) and FFB solution (approximately 84%) with the same composition as above.

The drug substance that was formulated was the product prepared according to the methods of examples 1 to 3.

### Preparation of Syn3/NODA Solution

The following steps were implemented to prepare the Syn3/NODA solution:
WFI was weighed and added to a single-use mixer bag to approximately 11% of final weight;
Hydroxypropyl-beta-cyclodextrin was weighed and added to the bag. The mixture was stirred until completely dissolved;
Syn3/NODA was weighed and added to the bag. The mixture was stirred until completely dissolved;
Citric acid monohydrate, trisodium citrate dihydrate, and WFI were weighed and added to a single-use flask to prepare a buffer solution, and the content was dissolved;
The buffer solution was weighed and added to the bag. The mixture was stirred until completely mixed;
Polysorbate 80 and WFI were weighed and added to a single use flask and the content is mixed. The content is thereafter added to the bag. The mixture was stirred until completely mixed;
WFI was added to final weight. The mixture was stirred until completely mixed;
The solution was filtered for bioburden reduction through a sterilising grade polyethersulfone 0.2 μm membrane filter and dispensed into bags;
The solution was stored at 2-8° C.

### Preparation of FFB Solution

The following steps were implemented to prepare the FFB solution:
WFI was weighed and added to a single-use mixer bag to approximately 88% of final weight;
Sodium dihydrogen phosphate dihydrate was weighed and added to the bag. The mixture was stirred until completely dissolved;
Tromethamine was weighed and added to the bag. The mixture was stirred until completely dissolved;
Sucrose was weighed and added to the bag. The mixture was stirred until completely dissolved;
Magnesium chloride hexahydrate was weighed and added to the bag. The mixture was stirred until completely dissolved;
Glycerol was weighed and added to the bag. The mixture was stirred until completely mixed.
pH was measured;
The solution was filtered for bioburden reduction through a sterilising grade polyethersulfone 0.2 μm membrane filter and dispensed into sterilised single-use bags;
The solution was stored at 15-25° C.

### Preparation of Blank Solution

This solution was used to pre-rinse the filter in sterile filtration:
Syn3/NODA solution was weighed and added to a single-use mixer bag;
FFB solution was weighed and added to the bag. Stirring was applied until completely mixed;
The solution was filtered for bioburden reduction through a sterilising grade polyethersulfone 0.2 μm membrane filter and dispensed into bags;
The solution was stored at 2-8° C.

### Formulation of Drug Product Suspension

The frozen drug substance bags stored below −60° C. were thawed by immersing in a water bath at 22.5-23.5° C. The drug product was then formulated according to the following steps:
the thawed drug substance was added to a 20 L single-use bag and the weight was recorded;
FFB solution was weighed and added to the bag;
Syn3/NODA solution was weighed and added to the bag;
the content of the bag was gently mixed by placing the bag on a rocker and mixed until homogenous.

This succession of steps, wherein Syn3/NODA solution was added the last, was the most optimal, as compared to other methods comprising, for example, mixing the drug substance to Syn3/NODA first. It results in a decrease of foam formation, and as a result, to a more streamlined process providing the most homogenous mixture of components.

The drug substance, nadofaragene firadenovec, is sensitive to heat and terminal sterilization could not therefore not be applied. The drug product suspension was thus manufactured through aseptic processing and sterilized by filtration through a sterilizing grade polyethersulfone 0.2 μm membrane filter, after a pre-rinse of the filter with the blank solution.

Vials could then be filled with the filtered drug product, and then be packaged and stored below −60° C.

The process disclosed herein advantageously provides an optimally stabilized drug product.

A study was performed on the drug product suspension manufactured as mentioned above, to assess stability outside of label storage conditions (−20° C.). This study provided assurance that the product remained stable for a suitable time period between thawing and administration.

Frozen drug product was thawed and monitored for on week at refrigerated conditions (+2-8° C.) and at room temperature (+20-25° C.). Stability was monitored with the following methods:
Appearance;
Viral particle concentration by AEX-HPLC;
Infectious titre;
Potency;
pH;
Particle size distribution.

Infectious titre, viral particle concentration and potency assays are able to detect virus degradation/loss, which has been confirmed by a forced degradation study.

All time points at both storage temperatures fulfilled the set acceptance criteria, and there were no differences between storage temperatures. Therefore, the drug product manufacture according to the present disclosure can be considered stable at room temperature and in the fridge for up to one week after thawing.

In addition, a freeze-thaw element was included in the study to confirm that the drug product retains its quality after one additional freeze-thaw step before use. In this part of the study, a vial of the drug product was thawed at room temperature and re-frozen the same day. The following day the vial was re-thawed and analyzed according to the methods above. There were no indications of adverse effect on the product supporting the possibility that vials can be re-frozen after thawing.

Thus, all the studies conducted show that the drug product manufactured as disclosed herein met the acceptance criteria for a product for administration to a human subject. They provide assurance that the product remains stable for a suitable period between thawing and administration, and for repeated freeze-thaw cycles.

1. A method for the purification of recombinant adenovirus vectors, comprising the comprising the steps of submitting a preparation of recombinant adenovirus vectors to two-step anion exchange chromatography, and recovering the recombinant adenovirus vectors from the two-step anion exchange chromatography elute.

2. The method according to claim 1, comprising the following steps:
a) submitting a recombinant adenovirus vector harvest to a first tangential flow filtration step to concentrate and condition the recombinant adenovirus vector;
b) submitting the concentrated and conditioned recombinant adenovirus vector to two-step anion exchange chromatography; and
c) submitting the recombinant adenovirus vector eluted from step b) to a second tangential flow filtration step.

3. The method according to claim 1, wherein the recombinant adenovirus vector was produced according to a production method which includes a step of lysing producer cells with a detergent.

4. The method according to claim 1, wherein the preparation of recombinant adenovirus vectors is a filtered bulk harvest.

5. The method according to claim 1, wherein both anion exchange chromatography steps are performed with strong anion exchangers.

6. The method according to claim 1, wherein the first anion exchange chromatography step is performed on a strong anion exchange membrane.

7. The method according to claim 1, wherein the second anion exchange chromatography step is performed on a strong anion exchange resin.

8. The method according to claim 1, wherein the recombinant adenovirus vector is a replication-incompetent adenovirus.

9. The method according to claim 1, wherein the recombinant adenovirus vector is a recombinant adenovirus 5 vector.

10. The method according to claim 1, wherein the recombinant adenovirus vector encodes an interferon.

11. The method according to claim 10, wherein the interferon is interferon α2b.

12. A method for the manufacture of a drug product, comprising the steps of: mixing a drug substance with a final formulation buffer (FFB) solution, and then with a Syn3/NODA solution, and (ii) filtering the resulting drug product with a sterilizing filter.

13. The method according to claim 12, wherein the drug substance is a recombinant adenoviral vector.

14. The method according to claim 12, wherein the manufactured drug product comprises:
about 3×10¹¹ vp/mL nadofaragene firadenovec,
about 0.95 mg/mL Syn3,
about 0.01 mg/mL citric acid monohydrate,
about 0.04 mg/mL Tri-sodium citrate dihydrate,
about 0.48 mg/mL polysorbate 80 (Tween 80),
about 7.9 mg/mL hydroxypropyl-beta-cyclodextrin,
about 1.4 mg/mL sodium dihydrogen phosphate dihydrate,
about 1.4 mg/mL tromethamine,
about 17 mg/mL sucrose,
about 0.34 mg/mL magnesium chloride hexahydrate,
about 84 mg/mL glycerol, and
Water (q.s.).
